(19) United States
(12) Patent Application Publication
(10) Pub. No.: US 2026/0317689 A1
(43) Pub. Date: Oct. 15, 2026

(54) SYSTEMS AND METHODS FOR BIOLOGICAL SEPARATORS

(71) Applicant: Life Technologies Corporation, Carlsbad, CA (US)

(72) Inventors: Brycen H. Mills, Millville, UT (US); Joshua L. Adams, Smithfield, UT (US); Mark T. Smith, Nibley, UT (US)

(21) Appl. No.: 19/483,876

(22) PCT Filed: May 10, 2024
(86) PCT No.: PCT/US2024/028959

§ 371 Date: Nov. 12, 2025

Related U.S. Application Data
(60) Provisional application No. 63/466,469, filed on May 15, 2023.
(60) Provisional application No. 63/472,919, filed on Jun. 14, 2023.

(51) Int. Cl. C12M 1/00; C12M 1/36

(52) U.S. Cl. CPC C12M 47/18; C12M 29/04; C12M 41/48

(57) ABSTRACT

The present invention relates to automated separation systems used for purifying multiphase fluid generated from bioreactors to prevent unintentional clogging of gas exhaust filters. In an embodiment, the separation system includes a separator assembly configured to generate a vortex motion for the multiphase fluid to efficiently break foam, release particle matter entrapped in the foam and release gases free of particle matter for passing through gas exhaust filters.

### CROSS-REFERENCE TO RELATED APPLICATIONS

This application claims the benefit of the U.S. Provisional Patent Application Ser. No. 63/466,469, filed May 15, 2023, and titled “SYSTEMS AND METHODS FOR BIOLOGICAL SEPARATORS, and U.S. Provisional Patent Application Ser. No. 63/472,919, filed Jun. 14, 2023, and titled “SYSTEMS AND METHODS FOR BIOLOGICAL SEPARATORS”.

### FIELD

The present disclosure relates to biological separator systems used for separating biological components from bioproduction systems. More specifically, the present disclosure is directed to biological separator systems for separating gases, liquids, and particles in exhaust gases, generated from bioproduction equipment.

### BACKGROUND OF THE INVENTION

Currently, bioprocessing equipment, including bioreactors and fermentors, is used to cultivate cells and microorganisms. They are used in various applications, including basic research and development (R & D) and the manufacturing of biopharmaceuticals, chemicals, and other products. One of the challenges in automating such bioprocessing equipment is overcoming the clogging of exhaust filters by exhaust gases, particles, or foam evolved during cell cultivation. Generally, exhaust gases are a mixture of unreacted purge gases, waste products from the cells, gaseous byproducts, moisture, foam, and/or particulate matter. When the exhaust gases are vented through exhaust filters, moisture condenses on the filters and entraps the particulate matter therein. Such situations call for frequent replacement of filters, adding cost and delay to the overall process and optimization.

Accordingly, what is needed in the art are time-efficient and cost-effective automated separator systems and methods for purifying exhaust gases and eliminating foam and particles from bioprocessing equipment that solve all or some of the above-identified shortcomings or other deficiencies known in the art.

### SUMMARY OF THE DISCLOSURE

It is understood that each independent aspect recited herein may include any of the features, options, and possibilities recited in association with the other independent aspects set forth above or as recited elsewhere within this document.

Example systems for separating multiphase fluid flows are herein disclosed. Multiphase fluid flows can include liquids, solids, gases, and foam. Example separator systems and assemblies for separating multiphase fluid flows can include a separator body having an upper portion and a lower portion, a chamber therein. A central axis extends from the upper portion to the lower portion of the separator body and through the chamber. An inlet is connected to and in fluidic communication with the separator body, chamber, and a fluid line that can be connected to fluid processing equipment, such as a bioreactor or a fermenter. The inlet is configured and designed to direct a multiphase fluid, including foam and entrapped particles, through the fluid line and tangentially into the chamber. A first outlet and a second outlet are connected to and in fluidic communication with the separator body and chamber and in fluidic communication with the inlet. An exhaust line has a first end and an opposing second end. The first end of the exhaust line is in fluidic communication with the first outlet, and the chamber of the separator body. The chamber of the separator body is configured to break foam and release particles entrapped therein.

In various embodiments, the spatial alignment of the inlet and the first and second outlets of the chamber is configured to flow a multiphase fluid tangentially into the chamber and/or flow the multiphase fluid in a vortex motion through the chamber. The chamber can further include a fluid pathway with a serpentine or spiral shape to facilitate the vortex or circular fluid flow around the central axis. The vortex motion of the multiphase fluid breaks foam in the fluid and separates liquids, solids, and gases. The gases free of solids, or gases substantially free of solids of a certain concentration or size, are routed and exhausted through the first outlet, and the solids and/or liquids are routed through the second outlet and into a holding tank positioned downstream from the separator body. The holding tank can be coupled to one or more sensors, including a foam sensor, level sensor, Raman sensor, and cell density sensor, to perform process analytics and compositional analysis of the contents of the holding tank.

In various embodiments, a method for separating a multiphase fluid in a separator is provided. The multiphase fluid can include gases, liquids, solids, and foam generated in a bioprocessing vessel. The separator can include a chamber with a central axis, an inlet, two outlets, and a fluid pathway connecting the inlet to the outlets. An inlet line can be connected between the outlet of the bioprocessing vessel and the inlet of the chamber to flow the multiphase fluid into the chamber of the separator. The inlet and/or inlet line can be configured to direct the multiphase fluid tangentially into the chamber, which can cause the foam to break and release gases, liquids, and solids. The fluid pathway in the chamber can also be shaped to cause the multiphase fluid to contact walls of the chamber, break the foam, and release gases, liquids, and solids in the foam. For example, the fluid pathway in the chamber can be formed, for example in a serpentine or spiral shape to cause the multiphase fluid to travel in a vortex motion through the chamber, breaking the foam and releasing gases, liquids, and solids from the foam. The arrangement and geometry of the inlet, outlets, and fluid pathway of the chamber can cause or route gases to move toward the central axis and out of the first outlet of the chamber and cause or route solids and liquids to move away from the central axis and out of a second outlet of the chamber. Gases routed through the first outlet of the chamber can be flowed through an exhaust line and into an exhaust unit with a filter for filtering out microbes from the gas. Liquids and/or solids routed through the second outlet can be flowed through a drain line and into a holding tank for analysis or disposal. The holding tank can be coupled to one or more sensors, including a foam sensor, level sensor, Raman sensor, and cell density sensor, to perform process analytics and compositional analysis of the contents of the holding tank and for feedback control.

In various embodiments, an automated bioprocessing system is provided that includes a system controller comprising a processor and memory for storing operational instructions and controlling components of the bioprocessing system. The bioprocessing system can include a first valve and a first pump in fluid communication with an outlet of a bioreactor or biofermentor that contains a multiphase liquid comprising gas, liquid, solid, and/or foam. A first sensor is coupled to the bioreactor or biofermentor and in electronic communication with the system controller. A first separator includes a chamber with an inlet port, a first outlet port, and a second outlet port in fluid communication with the outlet of the bioreactor or the biofermentor. A first set of instructions is stored in the memory of the system controller for controlling the pump and the valve and flowing the multiphase fluid comprising through the chamber to break foam and separate the gas from the solid. The chamber comprises a fluid pathway that routes the gas through the first outlet port and the solid through the second outlet port. The sensor can be a foam sensor and measurements from the foam sensor can be sent to the system controller for processing and determining when the multiphase fluid should be flowed into the separator to break foam and separate gas, liquid, and/or sold phases of the foam.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1A is a block diagram of an automated bioprocessing system, including a separator system in accordance with example embodiments.

FIG. 1B is a schematic view of the separator system in electronic communication with a system controller in accordance with example embodiments.

FIG. 2 is a perspective view of a separator assembly in accordance with example embodiments.

FIG. 3A is a front cross-sectional view of a separator assembly in accordance with example embodiments.

FIG. 3B is a side cross-sectional view of the separator assembly shown in FIG. 3A, and in accordance with example embodiments.

FIG. 3C is a top cross-sectional view of a separator assembly shown in FIG. 3A, and in accordance with example embodiments.

FIG. 4A is a schematic side view of a separator assembly in accordance with example embodiments.

FIG. 4B is a schematic top view of a separator assembly shown in FIG. 4A, and in accordance with example embodiments.

FIG. 5 is a flow diagram of a method for operating a separator system in accordance with example embodiments.

FIG. 6 is a block diagram of an example computing device that can automate all operations of an example automated bioprocessing system, including a separator system.

### DETAILED DESCRIPTION OF THE PREFERRED EMBODIMENTS

Before describing the present disclosure in detail, it is to be understood that this disclosure is not limited to particularly exemplified apparatus, systems, methods, or process parameters that may, of course, vary. It is also to be understood that the terminology used herein is only for the purpose of describing particular embodiments of the present disclosure and is not intended to limit the scope of the disclosure in any manner.

All publications, patents, and patent applications cited herein, whether supra or infra, are hereby incorporated by reference in their entirety to the same extent as if each individual publication, patent, or patent application was specifically and individually indicated to be incorporated by reference.

The term “comprising” which is synonymous with “including,” “containing,” or “characterized by,” is inclusive or open-ended and does not exclude additional, unrecited elements or method steps.

It will be noted that, as used in this specification and the appended claims, the singular forms “a,” “an” and “the” include plural referents unless the content clearly dictates otherwise. Thus, for example, a reference to a “partition” includes one, two, or more partitions.

As used in the specification and appended claims, directional terms, such as “top,” “bottom,” “left,” “right,” “up,” “down,” “upper,” “lower,” “proximal,” “distal” and the like are used herein solely to indicate relative directions and are not otherwise intended to limit the scope of the disclosure or claims.

Where possible, like numbering of elements has been used in various figures. Furthermore, multiple instances of an element and or sub-elements of a parent element may each include separate letters appended to the element number. For example, two instances of a particular element “10” or two alternative embodiments of a particular element may be labeled as “10a” and “10b”. In that case, the element label may be used without an appended letter (e.g., “10”) to generally refer to all instances of the element or any one of the elements. Element labels, including an appended letter (e.g., “10a”) can be used to refer to a specific instance of the element or to distinguish or draw attention to multiple uses of the element. Furthermore, an element label with an appended letter can be used to designate an alternative design, structure, function, implementation, and/or embodiment of an element or feature without an appended letter. Likewise, an element label with an appended letter can be used to indicate a sub-element of a parent element. For instance, an element “12” can comprise sub-elements or surfaces “12a” and “12b.”

Various aspects of the present devices and systems may be illustrated by describing components that are coupled, attached, and/or joined together. As used herein, the terms “coupled”, “attached”, and/or “joined” are used to indicate either a direct connection between two components or, where appropriate, an indirect connection to one another through intervening or intermediate components. In contrast, when a component is referred to as being “directly coupled”, “directly attached”, and/or “directly joined” to another component, there are no intervening elements present. Furthermore, as used herein, the terms “connection,” “connected,” and the like do not necessarily imply direct contact between the two or more elements.

Various aspects of the present devices, systems, and methods may be illustrated with reference to one or more example embodiments. As used herein, the term “embodiment” means “serving as an example, instance, or illustration,” and should not necessarily be construed as preferred or advantageous over other embodiments disclosed herein.

Unless defined otherwise, all technical and scientific terms used herein have the same meaning as commonly understood by one of ordinary skill in the art to which the present disclosure pertains. Although one or more methods and materials similar or equivalent to those described herein can be used in the practice of the present disclosure, the preferred systems, materials, and methods are described herein.

Example separator systems herein disclosed can include but are not limited to particle separator systems, foam separator systems, multiphase fluid separator systems, and combinations of the same. Example separator systems can be used for purifying exhaust gases and eliminating solids, particles, and/or dispersing foam from fluid and bioprocessing equipment, including but not limited to reactors, fermentors, mixers, bioprocess containers, filters, fluid storage containers, bubble traps, conduits, pumps, valves, and/or other fluid and bioprocessing equipment used to process, store, or flow biological fluids.

Based on the biological process, cell types, reactants, gases purged, and other physical parameters, including impeller speed, temperature, pressure, and humidity, a gaseous mixture of varying composition can be generated above a cell suspension and within bioprocessing equipment or associated bioprocess containers. The gaseous mixture can include a homogenous or heterogenous mixture of gaseous byproducts, moisture, foam, solids and/or particulate matter. In particular, foam bubbles are formed by the entrapment of the gaseous matter in liquid layers. Further, the bubbles tend to entrap particulate matter. With the progress of a biological process (e.g., cell or biocomponent cultivation process), the foamy gaseous matter starts to occupy a greater volume of the bioprocess container space. To conform to safety protocols and avoid the build-up of excessive pressure (e.g., exceeding a threshold pressure value) in the bioprocess container or other equipment, this gaseous matter must be exhausted frequently. An exhaust line connected to the gas outlet is used to discharge or exhaust the gaseous mixture from the bioprocessing equipment and/or associated bioprocess container and route it to one or more exhaust filters. The filter(s) purifies the exhaust gases and vents out the gasses free of particulate matter and/or harmful biological matter. But during this process, the filter can get clogged. As solids or particulate matter increase in concentration during processing (e.g., cell or biological component cultivation), the filters are clogged more frequently, and the process operation needs to be stopped to replace the filters. The example automated separator systems herein disclosed are configured to address eliminate, reduce, and/or disperse foam, solids, and/or particles to address the above complications and problems.

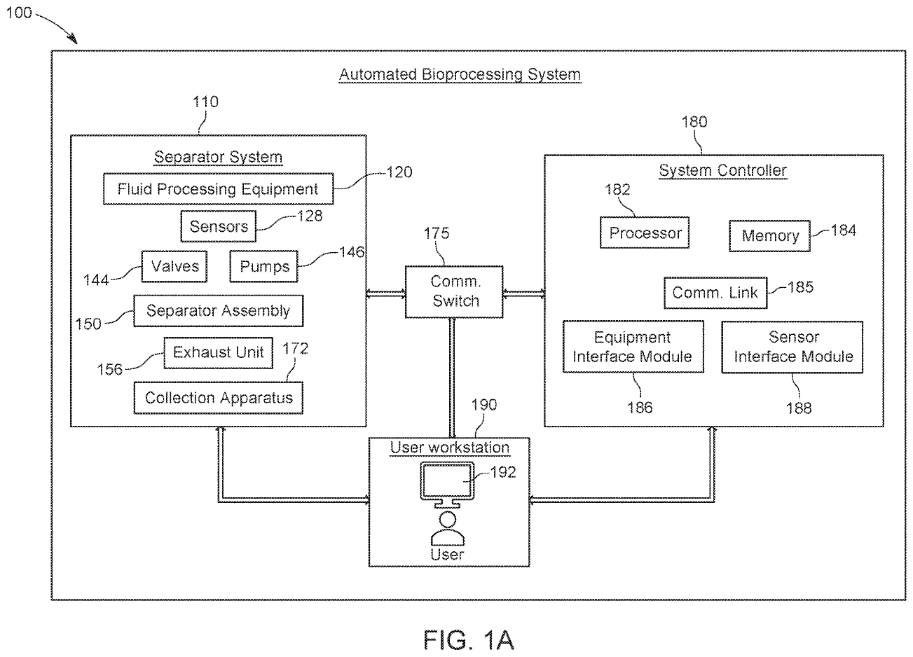

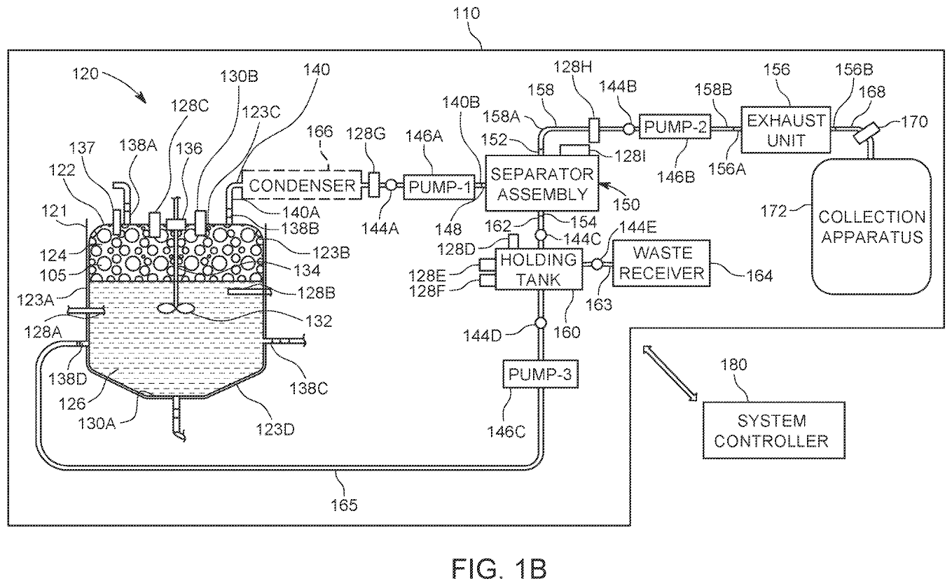

FIG. 1A is a block diagram of an automated bioprocessing system 100, consistent with implementations of the current subject matter. In general, bioprocessing system 100 is designed to purify exhaust gases and eliminate, reduce, and/or disperse foam, solids, and/or particles generated from a specific piece of fluid processing equipment to prevent unintentional clogging of the gas exhaust filters. Automated bioprocessing system 100 can include a separator system 110 (also referred to as a particle and foam separator system) and associated sensors 128 in electronic communication with a system controller 180, and a user workstation 190 operated by a user. A communication switch 175, for example, an ethernet IP switch, functions as a router to facilitate and balance communications and data transmission between separator system 110 and/or sensor 128 and sensor transmitter (not shown) associated with the system, system controller 180, and user workstation 190. Other communication links, routers, or switches can also facilitate and balance communications and data transmission between separator system 110, associated equipment, sensors 128, system controller 180, and user workstation 160.

Separator system 110 can include a fluid processing equipment 120, a plurality of sensors 128, a plurality of valves and/or associated manifolds 144, a plurality of pumps 146, a separator assembly 150, an exhaust unit 156, a collection apparatus 172 and/or other peripherals, instruments, and bioprocessing equipment. For the purposes of this example, the fluid processing equipment 120 is a bioreactor 120, but can be a fermentor, mixer, bioprocess container, storage tank, filter, pump, conduit or other bioprocessing equipment used to process biological or flow fluids. In an example embodiment, the fluid processing equipment 120 in separator system 110 can be a Thermo Scientific™ DynaDrive™ Single-Use Bioreactors (S.U.B.s), Thermo Scientific™ Single-Use Fermentor (S.U.F.), or any similar bioprocessing system.

Operations of the separator system 110, including the running of reactor 120, can be controlled by a system controller 180. Operation of one or more valves 144 and pumps 146 can be controlled by analog or digital input modules, transmitters, communication hubs, communication channels, and/or other signal and data communication and/or processing devices for processing and exchanging data with the controller (e.g., Ethernet/IP codesys, DI/DO modules).

System controller 180 includes at least one processor 182 and at least one associated primary memory 184 for storing instructions, which, when executed by at least one processor 182, are configured to perform one or more operations, including process control operations for automating fluid or bioprocessing equipment. Further, a communication link 185 facilitates electronic communication between separator system 110, sensors 128, valves 144, pumps 146, separator assembly, exhaust unit 156, collection apparatus 172, system controller 180, and user workstation 190, via communication switch 175. Communication link 185 can include any wired and/or wireless network, including, for example, a wide area network (WAN), a local area network (LAN), a virtual local area network (VLAN), a public land mobile network (PLMN), the Internet, and/or the like. All data interactions, including sending, receiving, writing, overwriting, and copying instructions, signals, and data between the above components, separator system 110, system controller 180, and user workstation 190, can be stored in memory 184.

In some implementations, memory 184 can be a centralized repository designed to store, process, and secure large amounts of structured, semi-structured, and unstructured data. In general, memory 184 can store and/or process the data received from separator system 110 and serve as a source of data for user workstation 190 and vice-versa. In various embodiments, portions of data stored in memory 184 can be configured to be transferred to plant or large-scale applications, while other portions of data can be used for bench-scale applications in a laboratory environment. For example, data stored in memory 184 can be used for data analytics, predictive protocols, and process optimization.

Further, system controller 180, includes an equipment interface module 186 and a sensor interface module 188, configured to generally interface with, receive and transmit signals and data to and from one or more operational components, peripherals, or equipment (valves 144, pumps 146) and sensors 128 (or associated transmitters) of the separator system 110. In various embodiments, the system controller 180 can be a single unit or a distributed control system with a client-side control component for client inputs and outputs and a plant-side control component closer in proximity to the bioprocessing plant.

A user can control operations of separator system 110 via user interface 192 displayed on user workstation 190. In particular, user interface 192, includes user inputs and readable instrument and process parameter outputs for controlling and monitoring separator system 110. For example, user workstation 190 can be configured to remotely control and monitor one or more operations of separator system 110 by receiving inputs/outputs from sensors 128, valves 144, pumps 146, separator assembly, exhaust unit 156, collection apparatus 172, other equipment, or associated transmitters.

FIG. 1B is a schematic view of separator system 110 in electronic communication with system controller 180 in accordance with example embodiments. More specifically, FIG. 1B depicts a reactor 120 in fluid communication with a downstream separator assembly 150. Reactor 120 is configured for biological reactions, including but not limited to, growing cells or other biological components. In example embodiments, bioreactor 120 can also comprise or be substituted with one or more bioreactors, fermenters, mixers, storage vessels, fluid management systems, cell culture equipment, centrifuges, centrifugal separators, chromatography units, mixers, homogenizers, magnetic processing units, blood separating devices, biocomponent filtering devices, biocomponent agitators or any other device designed for growing, mixing or processing cells and/or other biological components. It is also appreciated that bioreactor 120 can comprise any conventional type of bioreactor, fermenter, or cell culture device such as a stirred-tank reactor, rocker-type reactor, paddle mixer reactor, or the like.

In the example embodiment depicted, bioreactor 120 comprises a container 122 bounding a chamber 124. Container 122 is supported by a rigid support housing 121 and has a left sidewall 123A, a right sidewall 123B, a top sidewall 123C, and a bottom sidewall 123D. Disposed within chamber 124 is a bioprocess fluid 126. Bioprocess fluid 126 typically comprises one or more biological components that include cells or microorganisms and a growth medium in which the cells or microorganisms are suspended and grown.

By way of example and not by limitation, bioprocess fluid 126 can include one or more biocomponents, including fluids, solids, mixtures, solutions, and suspensions including, but not limited to, bacteria, fungi, algae, plant cells, animal cells, white blood cells, T-cells, cell media, protozoans, nematodes, plasmids, viral vectors, blood, plasma, organelles, proteins, nucleic acids, lipids, plasmids, carbohydrates, and/or other biological components, and the like. Examples of some common biological components that are grown in bioprocess fluid 126 and bioreactor 120 include E. coli, yeast, bacillus, and CHO cells. Bioprocess fluid 126 can also comprise cell-therapy cultures and cells and microorganisms that are aerobic or anaerobic and adherent or non-adherent. Different media compositions known in the art can be used to accommodate the specific cells or microorganisms grown and the desired end product. In some uses, bioprocess reactor 120 primarily grows and recovers cells for subsequent use (e.g., preparing vaccine materials from the cells themselves). But in many uses, the ultimate purpose of growing cells in bioprocess reactor 120 is to produce and later recover biological products (such as recombinant proteins, viral vectors, etc. . . . ) that are exported from the cells into the growth medium. It is also common to use bioprocess reactor 120 to grow cells in a master batch to prepare a specific volume, density, concentration, CFU, and/or aliquot of cells for subsequent use as an inoculant for multiple subsequent batches of cells grown to recover biological products.

In example embodiments, container 122 can comprise a flexible, collapsible bag. For example, container 122 can comprise one or more sheets of a flexible, water-impermeable polymeric film such as low-density polyethylene. It is appreciated that container 122 can be manufactured to have virtually any desired size, shape, and configuration. For example, container 122 can be formed having chamber 124 sized to 0.5 liters, 1 liter, 5 liters, 10 liters, 30 liters, 50 liters, 100 liters, 250 liters, 500 liters, 750 liters, 1,000 liters, 1,500 liters, 3,000 liters, 5,000 liters, 10,000 liters or other desired volumes. The size of chamber 124 can also be in the range between any two of the above volumes. In other embodiments, chamber 124 can have a larger or smaller volume. Although in the above-discussed embodiment, container 122 is described as a flexible, collapsible, bag, in alternative embodiments, it is appreciated that container 122 can comprise any form of a collapsible container or semi-rigid container. In some embodiments, container 122 can comprise a rigid container, such as comprised of metal, molded plastic, or a composite. Support housing 121 can be eliminated in this embodiment as container 122 is self-supporting.

In one exemplary embodiment of the present disclosure, means are provided for mixing the bioprocess fluid 126 within container 122. In the depicted embodiment, a movable mixing element 132 is disposed within chamber 124 and is used for mixing bioprocess fluid 126. In example embodiments, mixing element 132 can comprise an impeller coupled with a drive shaft 134. Drive shaft 134 couples with container 122 through a dynamic seal 136. A motor can be coupled with a drive shaft 134 for rotating mixing element 132 to facilitate the mixing of bioprocess fluid 126. Further, system controller 180 can control operation of the motor to control the speed of mixing element 132.

A bottom sparger 130A can be either positioned on or mounted to bottom sidewall 123D of container 122 for delivering gas to the fluid within container 122. Various gases are typically required in the growth of cells or microorganisms within container 122. The gas typically comprises air that is selectively combined with oxygen, carbon dioxide, and/or nitrogen. If needed, a top sparger 130B can be coupled to top sidewall 123C of container 122 for delivering gas to bioprocess fluid 126 within chamber 124. Each sparger 130A, 130B can be connected to their respective gas sources and the opening and closing of valves (not shown in FIG. 1B) connected in line with gas sources can be controlled by system controller 180.

A plurality of ports 138A-D is coupled with container 122 for delivering material into or removing material from chamber 124. By way of example and not by limitations, port 138A is coupled to top sidewall 123C and can be an inlet port for introducing material into chamber 124; port 138B is also coupled to top sidewall 123C and can be the exhaust port to vent out exhaust gases from chamber 124, and in fluid communication with separator assembly 150; port 138C is coupled to right sidewall 123B and can be an outlet port to remove material from chamber 124 for further processing or storage; and port 138D is coupled to left sidewall 123A and can be an inlet port for introducing material (for example recycled material) into chamber 124. Although only four ports 138A-D are shown, it is appreciated that container 122 can be coupled with any desired number of additional ports similar to ports 138A-D and that additional ports similar to ports 138A-D can be coupled at any desired location on container 122 such as left sidewall 123A, right side wall 123B, and top side wall 123C. Ports 138 A-D can be of the same configuration or different configurations and can be used for a variety of different purposes. For example, ports 138A-D can be coupled with fluid lines for delivering media, cell cultures, and/or other components into and out of container 122. Ports 138A-D can also be used for coupling probes to container 122. For example, when container 122 is used as a bioreactor for growing cells or microorganisms, ports 138A-D can be used for coupling probes such as temperature probes, pH probes, dissolved oxygen probes, and the like. Examples of ports 138A-D and how various probes and lines can be coupled thereto are disclosed in the United States Patent Publication No. 2006-0270036, published Nov. 30, 2006 and United States Patent Publication No. 2006-0240546, published Oct. 26, 2006, which are incorporated herein by specific reference. Ports 138A-D can also be used for coupling container 122 to secondary containers and to other desired fittings. It is noted that bioreactor 120 is not necessarily drawn to scale with regard to separator 150. Chamber 124 of bioreactor 110 will commonly have a fluid capacity of at least 3, 5, 10, 20, 50, 100, 200, or more times the fluid capacity of separator 150.

As needed, sensors 128A-C can be coupled with container 122 for detecting the properties of bioprocess fluid 126. By way of example and not by limitation, sensor 128A can be a temperature sensor, sensor 128B can be a pH sensor, and sensor 128C can be a foam sensor. Sensors 128A, 128B, and 128C can be coupled to left, right and top sidewalls 123A, 123B, and 123C, respectively. In other examples, 128A-C can comprise CO₂ sensors, oxygen sensors, pressure sensors, level sensors, Raman sensors, cell density sensors, mass spectroscopic measurements, and the like, and coupled to side walls 123A-C in different configurations. Each sensor 128A, 128B, and 128C can be in electronic communication with system controller 180 to provide feedback on their respective sensor measurements.

For example, at least one of the sensors 128C can continuously take measurements to allow continuous monitoring and control of foam levels (e.g., foam thickness, foam volume) in chamber 124. The foam levels can be automatically controlled based on continuous measurements. For example, system controller 180 can receive continuous, timed, or prompted measurements from sensor 128C within proximity of the liquid and/or foam to determine a foam layer thickness or volume formed in chamber 124 during processing and/or sparging. Furthermore, system controller 180 can assist with controlling the amount of foam in the container 122 by controlling delivery of anti-foam solution into chamber 124 by an antifoam dispensing device 137 coupled to top side wall 123C of container 122. System controller 180 can implement and deploy other foam control measures, including but not limited to reducing the flow rate of gas flowing through sparger 130A, 130B; modifying the power to a motor; modifying the rotational speed of the drive shaft 134, and mixing element 132, modifying the angle or depth of shaft 134 and mixing element 132 in the container 122/chamber 124; and/or reducing the feed flow rate into the container 122/chamber 124.

In the above-described embodiment, during the cultivation of biological components in bioprocess fluid 126, a multiphase process fluid 105, including gases, liquids, solids, and particulates, such as foam with entrapped gasses, liquids, and solids, is formed above bioprocess fluid 126. Generally, to oxygenate cells/microorganisms within bioprocess fluid 126 and to otherwise regulate the chemistry within bioprocess fluid 126, gas is sparged into bioprocess fluid 126 through spargers 130A, 130B while bioprocess fluid 126 within container 122 is being mixed, such as through impeller 132. An antifoam or surfactant can be added to the bioprocess fluid 126 by antifoam dispenser 137 to limit unwanted shear forces on the cells or microorganisms caused by the impeller or other mixing element. The sparged gassed bubbles pass up through bioprocess fluid 126 and combine into multiphase process fluid 105. The multiphase process fluid 105 passes out through outlet port 138B and eventually exits into the environment through exhaust unit 156 described below. As discussed below, if needed, the multiphase process fluid 105 can pass through condenser 166 before passing through exhaust unit 156. Regardless of antifoam, the combination of the surfactant, the waste from the cells/microorganisms, and/or the sparging bubbles passing through the culture, can cause the foam to progressively can build above bioprocess fluid 126 to create the multiphase process fluid 105 including foam. If the foam is left unchecked, it will eventually pass out through outlet port 138B as a multiphase process fluid 105, where it can enter and clog exhaust unit 156. Once the filter in exhaust unit 156 becomes clogged by the multiphase process fluid 105, the entire reactor system becomes inoperable, and the system needs to be shut down. As such, cells within bioprocess fluid 126 in container 122 die. The multi-phase process fluid 105 can also produce buildup and blockage within condenser 166 and can build up on other process components downstream of outlet port 138B. Examples of automated separator systems described below are configured to address the above complications and problems.

In the system depicted in FIG. 1B, container 122 is in fluid communication with separator assembly 150 by a fluid line 140 extending from exhaust port 138B to an inlet port 148 of separator assembly 150. In particular, fluid line 140 includes a first end 140A coupled to outlet port 138B of container 122, and a second end 140B coupled to inlet port 148 of separator assembly 150. Further, fluid line 140 can be fluid coupled with valve-1 144A and pump-1 146A to control fluid flow through fluid line 140. Separator assembly 150 has a first outlet port 152 and a second outlet port 154. First outlet port 152 of separator assembly 150 is in fluid communication with an exhaust unit 156 by a fluid line 158. In various embodiments, exhaust unit 156 can consist of a filter and/or housing to house a filter (e.g., bacterial/viral, membrane, cartridge, and/or heat and moisture exchange filters) capable of purifying exhaust gas from biological and other processes.

In particular, fluid line 158 includes a first end 158A coupled to first outlet port 152 of separator assembly 150, and an opposing second end 158B coupled to exhaust unit 156. Further, fluid line 158 can be fluid coupled with valve-2 144B and with pump-2 146B to control fluid flow through fluid line 156. Likewise, second outlet port 154 of separator assembly 150 is in fluid communication with a holding tank 160 by a fluid line 162. Further, fluid line 162 can be fluid coupled with valve-3 144C to control fluid flow through fluid line 162. In some examples, pump-1 146A, and pump-2 146B may be optional and used in case of excessive pressure drop across respective fluid lines and or equipment. Each of valves 144A-C, pumps 146A-B, and associated transmitters are in electronic communication with system controller 180, and the operation of valves 144A-C and pumps 146A-B can be controlled by system controller 180. Also, fluid lines 140, 158, 162, and other fluid lines discussed herein can comprise flexible, polymeric tubing that can be coiled without plastic deformation. However, the fluid lines can comprise other flexible or rigid conduits in other embodiments.

Multiphase process fluid 105 can include significant gas and flow as exhaust out of bioreactor 120. For example, multiphase process fluids 105 or exhaust streams can include solids, liquids, and gases, such as foam and/or foam with entrapped liquids, solids, and particulates. Separator assembly 150 as described herein, is configured to process multiphase process fluids 105 from bioreactor 120 to (i) break down foam and release solids and particulate matter, (ii) separate a light phase (e.g., gas) from a heavy phase (e.g., liquid and solids), allowing gasses free of liquid and solids to vent out of first outlet 152, and (iii) facilitate collection of the heavy phase, including liquids, solids and particle matter from second outlet 154. The light phase, including gases venting out of first outlet 152 can include, but are not limited to oxygen, nitrogen, and air. The heavy phase with liquids, solids, and/or particulate matter collected from second outlet 154 can include but is not limited to organic or inorganic solids and liquids, water, cell media components, biological liquids, biological solids, biological debris, such as cell debris, surfactants, proteinaceous materials, poloxamer materials (e.g., poloxamer 188), PEG, algae-based materials, biologicals products, or other solids and liquids depending on type of process and reaction within bioreactor 120.

Gases venting out of first outlet 152 pass through an exhaust unit 156, including an inlet port 156A and an outlet port 156B. In example embodiments, exhaust unit 156 includes a filter, such as a sterilizing filter that can remove contaminates down to 0.2 microns. Other filters can also be used. More specifically, filters in exhaust unit 156 comprise a porous material through which gas can pass but through which unwanted contaminants, such as bacteria and microorganisms, cannot. The porous material is typically hydrophobic, which helps it to repel liquids. For example, the filter can be comprised of a polyethersulfone (PES) membrane or polyvinylidene fluoride (PVDF). Other materials can also be used. Commonly, the porous material has a pore size in a range between 0.22 and 0.18 In still other applications, the pore size can be greater than 1.0 μm for example, the DURAPORE™ 0.22 μm hydrophobic cartridge filter produced by Millipore. Another example is the PUREFLO UE™ cartridge filter available from ZenPure. Furthermore, exhaust unit 156 is fluid coupled with a collection apparatus 172 by a fluid line 168. A particle counter 170 can be interposed on fluid line 168 to sense/count a number of particles in gases exhausted out of exhaust unit 156. Collection apparatus 172 typically comprises a container configured for the safe receipt of exhaust gases and storage until the gases can be processed and/or released safely into the external environment.

The heavy phase of the multiphase process fluid 105 fed into the separator assembly 150, including solids, particle matter, biological product, cell media and other solid and liquid heavies, collected from the second outlet 154 is flowed down to holding tank 160 by opening of valve 144C. Holding tank 160 is coupled to one or more sensors for measuring parameters of the contents of the heavy phase received from second outlet 154 of separator assembly 150. In example embodiments, sensor 128D coupled to holding tank 160 can be a foam sensor to measure the foam volume, thickness, or concentration in holding tank 160; sensor 128E can be a level sensor for measuring liquid levels in holding tank 160; and sensor 128F can be a Raman sensor, cell density sensor or include a probe for allowing mass spectroscopy measurements for identifying the structural composition of the heavy phase collected in holding tank 160. The measurements from sensors 128D-F coupled to the holding tank can be used as feedback to system controller 180 to determine if foam-out is occurring or if product from the heavy phase in holding tank 160 needs to be recycled back to bioreactor 120 by opening a valve 144D and operating pump-3 146C via fluid line 165. If foam-out is occurring, measurements from foam sensor 128D can trigger system controller 180 (FIG. 1A) to deploy and cause a foam reduction measure (e.g., deploying antifoam or reducing gas flow, feed flow, or mixing speed). Holding tank 160 can also include a waste line 163 fluidly connected to waste receiver 164. A valve 144E fluidly coupled to waste line 163 can be opened by system controller 180 if recycling of components of the heavy phase is not needed. Holding tank 160 can be comprised of rigid material and/or the same type of materials as discussed above with regard to container 122 of bioreactor 120.

Other sensors 128G-H can be added to fluid lines 140, 158, to sense and measure fluid flow through different stages of separator system 110. For example, a flow sensor 128G measures fluid flow coming out from outlet port 138B of bioreactor 120; a flow sensor 128H measures fluid flow coming out of first outlet port 152 of separator 150. All sensors 128A-H and/or associated transmitters are in electronic communication with sensor interface module 188 of system controller 180. For example, each of sensors 128A-H is configured to transmit a signal and/or measurements to system controller 180 indicating various physical/fluid parameters or when measured physical parameters equal, exceed or fall below a threshold value.

Further, in some embodiments, sensor 128I is a foam sensor, and can be coupled to separator assembly 150 as shown in FIG. 1B, to sense foam volume, foam thickness, foam height, or foam concentration during a separation process in the separator assembly 150. In other embodiments, sensor 128I can be disposed proximate to bioreactor 120 or separator assembly 150 by coupling directly or indirectly to fluid line 140. In some other embodiments, sensor 128I can be coupled directly or indirectly to fluid line 158 to monitor foam levels in exhaust gases evolving out of first outlet port 152 of separator assembly 150. Sensor 128I and/or associated transmitters are in electronic communication with sensor interface module 188 of the system controller 180. Sensor 128I is configured to transmit a signal and/or measurements to system controller 180 indicating when a foam parameter like foam volume, thickness, or concentration equal, exceeds, or falls below a threshold value in separator assembly 150. In some examples, sensor 128I can include radar transmitters, pressure differential transmitters, dielectric differential transmitters, and/or ultrasonic transmitters. Further, system controller 180 can be configured to simultaneously monitor and receive feedback from foam sensor 128C coupled to bioreactor 120, foam sensor 128D coupled to holding tank 160, and foam sensor 128I coupled to separator assembly 150.

As shown in FIG. 1B, if desired, a condenser 166 can be interposed on fluid line 140, between exhaust port 138B of container 122 and separator assembly 150, so that multiphase process fluid 105 passes through condenser 166. Condenser 166 can be used to remove moisture from the multiphase process fluid 105 before it reaches separator assembly 150. Under certain processing conditions, the condensed moisture can cause a phase change of one or more components of the multiphase process fluid 105 creating ice or frozen particles based on a temperature of condenser 166 and other system parameters. Further, such frozen particles produced in condenser 166 can be separated by separator assembly 150 described herein. Optionally condenser 166 can be interposed on fluid line 158, between separator assembly 150 and exhaust unit 156 to condense any moisture from the exhaust gases before they enter the exhaust unit 156. One example of a condenser that can be used as condenser 166 and the remaining components needed to operate the condenser 166 are disclosed in U.S. Pat. No. 8,455,242, which was issued on Jun. 4, 2013, and which is incorporated herein in its entirety by specific reference. Another example of condensers that can be used is disclosed in the U.S. patent application Ser. No. 14/588,063, filed Dec. 31, 2014, which is incorporated herein in its entirety by specific reference. Other condensers and filters can also be used.

In various example embodiments, a condenser is not needed in separator system 110, and separator assembly 150 can handle all light and heavy phase separations without the need to condense components of multiphase process fluid 105.

In example embodiments, a plurality of separator assemblies 150 can be connected in a series or parallel configuration to separate a light phase from a heavy phase of the multiphase process fluid 105, enhance the purification of multiphase process fluid 105 and reduce foam generated from bioreactor 120. In a series configuration, gas exhaust outlet 138B from bioreactor 120 is connected to inlet 148 of a first separator assembly 150, gas exhaust outlet 152 from first separator assembly 150 can be connected to inlet 148 of a second separator assembly 150, gas exhaust outlet 152 from second separator assembly 150 can be connected to inlet 148 of a third separator assembly 150 and so on until a desired particle separation is achieved. Whereas in a parallel configuration, gas exhaust outlet 138 from bioreactor 120 can be connected to inlets 148 of a plurality of separator assemblies 150 connected in parallel gas exhaust outlets 152 from the plurality of separator assemblies connected in parallel can be connected to a common exhaust unit 156.

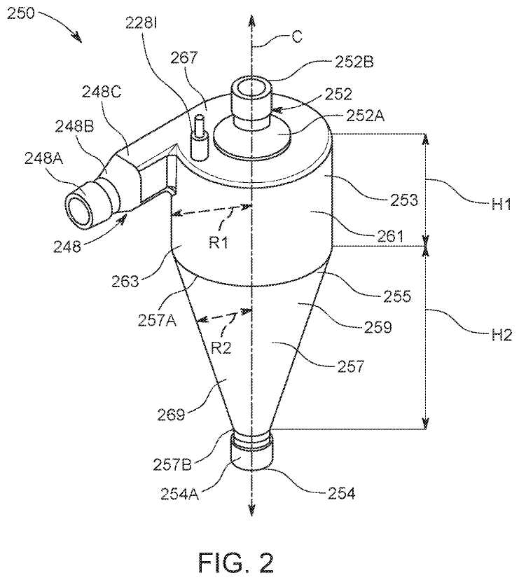

FIG. 2 illustrates a perspective view of an isolated separator assembly 250. Separator assembly 250 is an example of separator assembly 150 and can be used as separator assembly 150 in conjunction with fluid processing equipment 120 and other system components and alternatives discussed above with regard to FIGS. 1A-1B. Separator assembly 250 can be used to effectively break down foam generated during a process and to separate heavy and light phases of a multiphase process stream (e.g., multiphase process fluid 105) entering the separator assembly 250. The multiphase process stream can comprise gases, foam, liquids, solids and particulates. Separator assembly 250 is designed to separate liquids, solids, and particulates from gases in the process stream, while at the same time break down, reduce, and/or eliminate foam generated during the process. The light phase (e.g., gases) of the process stream can exit a first port 252 near the top of the separator assembly 250, and the heavy phase (e.g., liquid, solids, particulates) of the process stream can exit a second port 254 near the bottom of the separator assembly 250. As the separator assembly 250 breaks down foam, the gas component of the foam exits the first port 252 and the liquid/solids components of the foam exits the second port 254. This is particularly vital in biological processes, such as cell cultivation, plasmid production, vaccine production, antibody production and other biological processes that use gasses and liquids, generate foam, and have sensitive biological products. Biological products, such as cells, vaccines, plasmids, and antibodies can be degraded or destroyed from antifoam, foam reduction measures, high shear, and other process equipment that is not configured to handle and process sensitive, multiphase biological components and processes that generate significant foam. Separator assembly 250 can eliminate the need for antifoam and foam reduction measures while reducing/eliminating foam and preserving the biological product.

In general, separator assembly 250 comprises a body assembly 255, including an upper portion 253, a lower portion 257, and a chamber 259 defined therein. A central axis C extends from upper portion 253 to lower portion 257 through chamber 259. Upper portion 253 and lower portion 257 can be integrally formed with, or releasably attached to each other. As discussed below in more detail, body assembly 255 of separator 250 includes an inlet port 248 configured to receive a flow of multiphase process streams from fluid processing equipment 120, a first outlet port 252 configured to exhaust a light phase of multiphase process stream consisting of gases, and a second outlet port 254 configured to flow a heavy phase of multiphase process stream consisting of liquids, solids and/or particulate matter. While inlet port 248 and first outlet port 252 are attached to upper portion 253, second outlet port 254 is attached to lower portion 257 of body assembly 255.

Upper portion 253 includes a cylindrical wall structure 261 having a height of H1, and a circular cross-section of radius R1. Further, cylindrical wall structure 261 includes a side end wall 263 and a top end wall 267. While inlet port 148 is attached to side end wall 263 in a direction tangential to cylindrical wall structure 261 of upper portion 253, first outlet port 252 is attached to top end wall 267 in a direction aligning to central axis C. As shown in FIG. 2, inlet port 248 is comprised of a hose fitting section 248A configured to attach to first end 139 of fluid line 140, a frustoconical middle section 248B, and a cylindrical end section 248C configured to attach to side end wall 263 of upper portion 253. Similarly, first outlet port 252 comprises a frustoconical end section 252A attached to top end wall of upper portion 253, and hose fitting section 252B configured to attach to first end 155 of fluid line 156. Optionally, inlet port 248 and outlet port 252 can include other geometries and configurations for their respective end and middle sections.

Lower portion 257 includes a frustoconical wall structure 269 having a height H2, and a circular cross-section of decreasing radius R2 in a direction away from upper section 253. Further, lower portion 257 comprises a first end 257A, proximate to upper section 253, and an opposite second end 257B, with first end, 257A having a radius R2 matching to radius R1 of upper section. Second outlet port 254 is attached to second end 257B of lower section 257, and is aligned to central axis C, similar to first outlet 252. As shown in FIG. 2, outlet port 254 comprises a hose section 254A configured to attach to holding tank 160. Optionally, outlet port 254 can include other geometries and configurations for end and middle sections.

Further, in some embodiments, sensor 228I is a foam sensor, and can be coupled to separator assembly 250 to sense foam volume, foam height, foam thickness, or foam concentration during a separation process in separator assembly 250. As shown in FIG. 2, sensor 228I is coupled to top wall 267 to measure foam level in upper portion 253 of separator assembly proximate to first outlet 252. In other non-limiting examples, sensor 228I can be coupled to lower portion 257 to measure foam level proximate to second outlet 254. In case of foam out in separator assembly 250, foam sensor 228I coupled to upper portion 253 or lower portion 257 can transmit signals to system controller 180 to trigger actions to control foam generation in reactor 120.

A variation of heights H1, H2, and radiuses, R1, R2 of upper portion 253, and lower portion 257 can help achieve different separation characteristics for multiphase process streams from fluid processing equipment 120. For example, a smaller radius for lower portion 257 can be advantageous for separating particulate matter based on their particulate size, whereas a bigger radius for lower portion 257 can be advantageous for breaking down foam in multiphase process streams from fluid processing equipment 120. In a first example, dimensions for separation assembly 250 for separating particulate matter based on their particulate size, can include R1=2.0 inches, H1=1.5 inches, and R2 tapered from 2.0 inches down to 0.5 inches with H2=3.25 inches. In a second example, dimensions for separation assembly 250 for breaking down foam can include R1=4 inches, and H1=3 inches. R2 tapered from 4.0 inches down to 1.0 inches, and H2=1.0 inches. In both examples above, geometries and configurations for inlets 248 and outlets 252, 254 can be varied to optimize particle separation targets.

Separator assembly 250 can be made from molding materials by using plastic molding techniques, including, injection molding, or 3D printing techniques. For example, materials suitable for injection molding of separator assembly 250 include High-density polyethylene (HDPE), Low-density polyethylene (LDPE), and Vectra Liquid Crystalline polymers (Vectra LCP). In other examples, materials suitable for 3D printing of separator assembly 250 include Stereolithography (SLA)-compatible resins like tough, durable, standard, and heat resistant resins. Separator assemblies herein disclosed 150, 250 can be used with fluid processing equipment 120 and other system components discussed above in FIGS. 1A-1B. For example, separator assembly 150, 250 can be used one time or multiple times for separating one or more batches of multiphase fluids generated from fluid processing equipment 120. In other examples, separator assembly 150, 250 can be used to separate multiphase fluids for a designated period of time, including 1 hour, 5 hours, 10 hours, 24 hours, 48 hours, 72 hours, 96 hours, 1 week, 2 weeks or various times in between prior to being replaced with another separator assembly 150, 250. In other examples, for a first batch of separation, a first separator assembly 150, 250 can be used and then removed by disconnecting from fluid lines 140, 154, and 158. Further, for a second batch of separation, the first separator 150, 250 can be replaced with a second separator assembly 150, 250 to avoid downtime, avoid cleaning the first separator and obtain better separation efficiencies in each batch. The separator assembly 150, 250 can be formed from rigid material, plastic, other polymer materials, 3D printed materials or other materials that make a first separator assembly 150, 250 disposable and easily removable and interchangeable with a second separator assembly 150, 250. Example embodiments provide for a single-use operation of separator assembly 150, 250 to reduce downtime and cleaning, and prevent overuse, breakage, malfunction, and sterilization issues. Hence, automated bioprocessing system 100, including separator system 110, provide options for both multiple-use or reuse and single-use operation of separator assembly 150, 250.

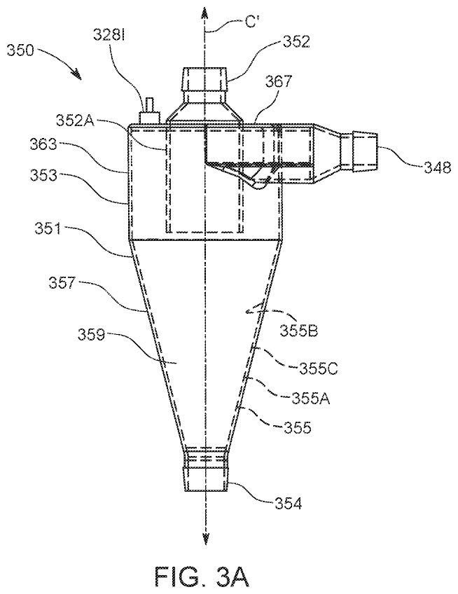

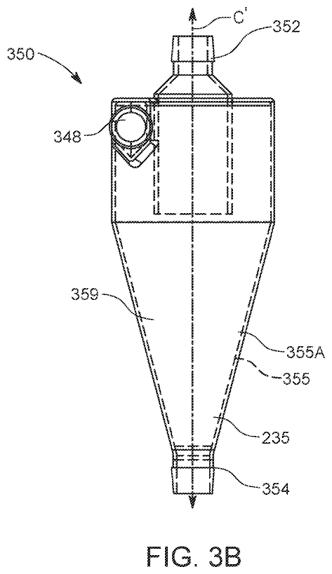

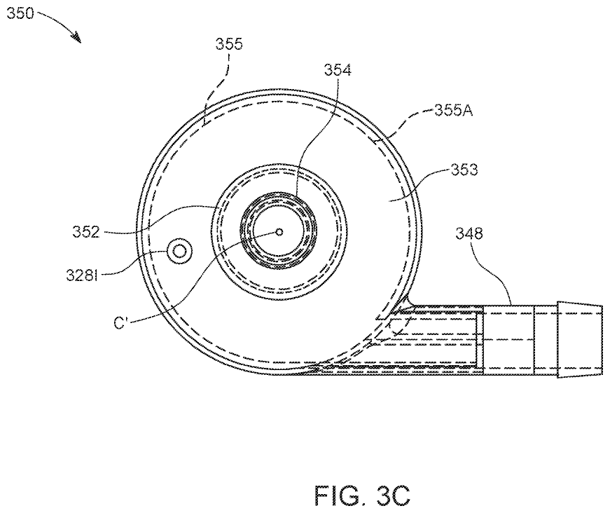

FIG. 3A illustrates a front cross-sectional view of another alternative embodiment for an isolated separator assembly 350. Separator assembly 350 is an example of separator assembly 150 and can be used as separator assembly 150 in conjunction with fluid processing equipment 120 and other systems components and alternatives discussed above with regard to FIGS. 1A-1B. Separator assembly 350 can be used to effectively break down foam generated during a process and to separate heavy and light phases of a multiphase process stream (e.g., multiphase process fluid 105) entering the separator assembly 350. The multiphase process stream can comprise gases, foam, liquids, solids and particulates. Separator assembly 350 is designed to separate liquids, solids, and particulates from gases in the process stream, while at the same time break down, reduce, and/or eliminate foam generated during the process. The light phase (e.g., gases) of the process stream can exit a first port 352 near the top of the separator assembly 350, and the heavy phase (e.g., liquid, solids, particulates) of the process stream can exit a second port 354 near the bottom of the separator assembly 350. As the separator assembly 350 breaks down foam, the gas component of the foam exits the first port 352 and the liquid/solids components of the foam exits the second port 354. This is particularly vital in biological processes, such as cell cultivation, plasmid production, vaccine production, antibody production and other biological processes that use gasses and liquids, generate foam, and have sensitive biological products. Biological products, such as cells, vaccines, plasmids, and antibodies can be degraded or destroyed from antifoam, foam reduction measures, high shear, and other process equipment that is not configured to handle and process sensitive, multiphase biological components and processes that generate significant foam. Separator assembly 350 can eliminate the need for antifoam and foam reduction measures while reducing/eliminating foam and preserving the biological product. Separator 350 can be a single-use alternative of separator 250, and like elements are identified by like reference characters. Furthermore, unless otherwise described and/or depicted, it is appreciated that like elements between separator 350 functions in the same way and can have the same alternatives as corresponding elements of separator 250 described above.

Separator assembly 350 includes a body support housing 351 supporting a body assembly 355. In part, body assembly 355 includes an upper portion 353, and lower portion 357, and a chamber 359 defined therein. A central axis C′ extends from upper portion 353 to lower portion 357 and through chamber 359. Body assembly 355 can also include inlet ports and one or more outlet ports through which fluid can flow into and out of chamber 359, respectively, as described in more detail below. For example, body assembly 355 includes an inlet port 348, and first outlet port 352 that is attached to upper portion 353, and second outlet port 354 that is attached to lower portion 357. First outlet port 352 includes a tubular extension 352A extending into upper portion 353 of body assembly 355, and interior to chamber 359.

In one embodiment, body assembly 355 comprises a flexible and collapsible bag 355A, having an interior surface 355B and an opposing exterior surface 355C, and interior surface 355B bounding chamber 359. In other words, body assembly 355 represents an internal liner layer, formed by flexible and collapsible bag 355A defining chamber 359, and housed by rigid support housing 351.

For example, body housing 351, and body assembly 355 can be comprised of the same type of materials as discussed above with regard to housing 121 and its respective container 122 of bioreactor 120. In addition, body housing 351 can be formed from rigid material configured to support body assembly 355, and body assembly 355 can be formed from a flexible material. Body assembly 355 can include flexible water-impermeable material such as polyethylene or other polymeric sheets having a thickness in a range between about 0.1 mm to about 5 mm, with about 0.2 mm to about 2 mm being more common. Other thicknesses can also be used. The flexible material can be comprised of a single-ply material or can comprise two or more layers that are either sealed together or separated to form a double wall body assembly. Where the layers are sealed together, the material can comprise a laminated or extruded flexible material. The laminated flexible material comprises two or more separately formed layers that are subsequently secured together by an adhesive.

The extruded material comprises a single integral sheet that comprises two or more layers of different materials that can be separated by a contact layer. All of the layers are simultaneously co-extruded. One example of an extruded material that can be used in the present invention is the Thermo Scientific CX3-9 film available from Thermo Fisher Scientific. The Thermo Scientific CX3-9 film is a three-layer, 9 mils cast film produced in a cGMP facility. The outer layer is a polyester elastomer coextruded with an ultra-low-density polyethylene product contact layer. Another example of an extruded material that can be used in the present invention is the Thermo Scientific CX5-14 cast film, also available from Thermo Fisher Scientific. The Thermo Scientific CX5-14 cast film comprises a polyester elastomer outer layer, an ultra-low density polyethylene contact layer, and an EVOH barrier layer disposed of therebetween. In still another example, a multi-web film produced from three independent webs of a blown film can be used. The two inner webs are each a 4.0 mil monolayer polyethylene film (which is referred to as the Thermo Scientific BM1 film), while the outer barrier web is a 5.5 mil thick 6-layer coextrusion film (which is referred to as the Thermo Scientific BX6 film).

The material is approved for direct contact with living cells and is capable of maintaining a sterile solution. In one embodiment, the material can be sterilizable such as by ionizing radiation or other conventional techniques. Other examples of materials that can be used in different situations are disclosed in U.S. Pat. No. 6,083,587 was issued on Jul. 4, 2000, and United States Patent Publication No. US 2003-0077466 A1, published Apr. 24, 2003, hereby incorporated by specific reference.

In one embodiment, body assembly 355 comprises a two-dimensional pillow-style bag wherein two sheets of material are placed in overlapping relation, and the two sheets are bounded together at their peripheries to form chamber 359. Alternatively, a single sheet of material can be folded over and seamed around the periphery to form chamber 359. In another embodiment, body assembly 355 can be formed from a continuous tubular extrusion of polymeric material that is cut to length and is seamed closed at the ends. In still other embodiments, such as in the depicted embodiment, body assembly 355 comprises a three-dimensional bag that has not only an annular side wall 363 but also a top-end wall 367. Three-dimensional containers can comprise a plurality of discrete panels, typically three or more, and more commonly four or six. Each panel can be substantially identical and comprise a portion of the side wall, and the top end wall of the container. The corresponding perimeter edges of each panel can be seamed. The seams are typically formed using methods known in the art, such as heat energies, RF energies, sonics, or other sealing energies.

In alternative embodiments, the panels can be formed in a variety of different patterns. Further disclosure with regard to one method of manufacturing three-dimensional bags is disclosed in United States Patent Publication No. US 2002-0131654 A1 published Sep. 19, 2002, of which the drawings and detailed description are hereby incorporated by reference.

Although in the above-discussed embodiment, body assembly 355 is in the form of a flexible bag, in alternative embodiments, it is appreciated that body 355 can also comprise any form of a collapsible container or semi-rigid container. Body assembly 355 can also be transparent or opaque and can have ultraviolet light inhibitors incorporated therein.

It is appreciated that body assembly 355 can be manufactured to have virtually any desired size, shape, and configuration. For example, body assembly 355 can be formed having chamber 359 that is sized to hold in a range from about 10 liters to about 2,000 liters, with about 20 liters to about 250 liters and about 20 liters to about 100 liters being more common. Other volume sizes can also be used. Although body assembly 355 can be any shape, in one embodiment body assembly 355 is specifically configured to be substantially complementary to chamber 359 defined by support housing 351.

Further, in some embodiments, sensor 328I is a foam sensor, and can be coupled to separator assembly 350 to sense foam volume, foam height, foam thickness, or foam concentration during a separation process in separator assembly 350. As shown in FIG. 3A, sensor 328I is coupled to top wall 367 to measure foam level in upper portion 353 of separator assembly proximate to first outlet 352. In other non-limiting examples, sensor 328I can be coupled to lower portion 357 to measure foam level proximate to second outlet 354. In case of foam out in separator assembly 250, foam sensor 328I coupled to upper portion 353 or lower portion 357 can transmit signals to system controller 180 to trigger actions to control foam generation in reactor 120.

FIG. 3B illustrates a side-cross-sectional view of separator assembly 350 with body assembly 355 including flexible bag 355A. Inlet 348 extends orthogonal to the plane of FIG. 3B and first and second outlets 352, 354 are aligned coaxially with central axis C′ extending through chamber 359. Likewise, FIG. 3C illustrates a top-cross-sectional view of separator assembly 350 with body assembly 355, including flexible bag 355A. Central axis C′ extends orthogonal to the plane of FIG. 3C, and inlet 348 is aligned tangential to upper portion 353 of body assembly 355, and first outlet 352, and second outlet 354 are aligned coaxially with central axis C′. In some examples, sensor 328I can be positioned opposite to inlet 348 to measure foam content in upper position 353 of separator assembly 350. Other outlet and inlet positions, angles, geometries, and configurations are also possible.

Separator assembly 350 can be used with fluid processing equipment 120 and other system components discussed above in FIGS. 1A-1B. For example, body assembly 355, constituting a lining of separator assembly 350, and the foam sensor 328I coupled to separator assembly 350 can be changed, removed, or replaced for each batch of separation of gases, liquids, and particles in exhaust gases, generated from fluid processing equipment 120 to provide a single-use operation of separator assembly 350. In other words, separator assembly 350 includes disposable body assembly 355. Such examples allow for the implementation of disposable technology to customize separator assembly 350 for each subsequent batch of separation.

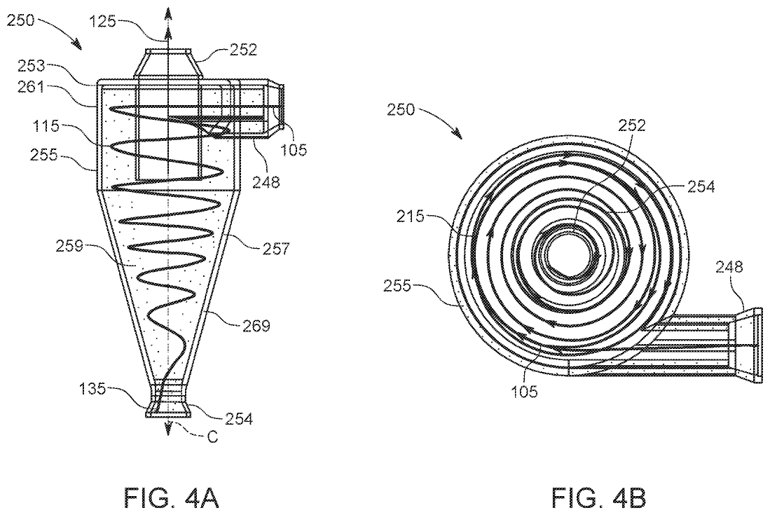

FIG. 4A illustrates a schematic side view of separator 250 described above and shows a fluid flow path for multiphase process fluid 105 exhausted from fluid processing equipment 120. It can be noted that the description provided below is in reference to separator 250, but a similar description can be envisaged for separator 350, as well. As discussed before, multiphase process fluid 105 flowed/exhausted from exhaust outlet 138B of bioreactor 120 comprises a mixture of gases, liquids, solids, particulate matter, and/or foam. As multiphase process fluid 105 enters body assembly 255 of separator 250 tangentially, a high-speed rotating fluid flow 115 is established within chamber 259. As mentioned before, foam sensor 128I coupled to upper portion 253 of separator assembly 250 provides for measuring foam height, foam concentration, and foam volume in the multiphase process fluid 105. One or more components of the multiphase process fluid 105 flows in a spiral or helical fluid path 115, beginning at upper portion 253 of chamber and ending at lower portion 257 before gases exiting chamber 259 in a straight stream 125 through the first outlet 252. The spiral fluid path 115 causes the process fluid 105 to flow in a vortex motion. The vortex motion forces foam layers and various phase and components of the multiphase process fluid 105 to swirl around and hit inner sides of cylindrical wall 261 and frustoconical side wall 269 repeatedly. The flow of foam through the separator assembly and against walls thereof disperses, disintegrates and/or breaks/breaks down—the foam and foam film layers, accelerates drainage, coalescence, and coarsening of the foam, disassociates gas, liquid and/or solid components of the foam and releases particles and gases entrapped in the foam. The lighter gas phase free of particle matter is concentrated around central axis C of chamber 259 and exhausted out through first outlet 252, and further through exhaust unit 156. The heavy phase consisting of denser liquid, solids and/or particle matter 135 released from foam along the fluid path 115 with higher mass and inertia cannot following the fluid path 115, strike the inner side of frustoconical wall 269, then fall towards second outlet 254 where they can be removed and collected in holding tank 160 described above. In a frustoconical lower portion, as the fluid path 115 moves towards the lower portion 257, the rotational radius of the fluid path 115 is reduced, thus separating smaller and smaller particles. The geometry of the body assembly 255 of separator 250 and the volumetric flow rate defines separation characteristics for separator assembly 250.

FIG. 4B illustrates a schematic top view of separator 250 shown in FIG. 4A. A spiral or helical fluid flow path 215 with a reducing radius for the helix of multiphase process fluid 105 exhausted from a bioreactor 120 is illustrated. An appropriate spatial alignment of the inlet 248, first outlet 252, second outlet 254, and geometry of body assembly 255, is crucial for generating a vortex motion for the fluid 105 entering into chamber 259.

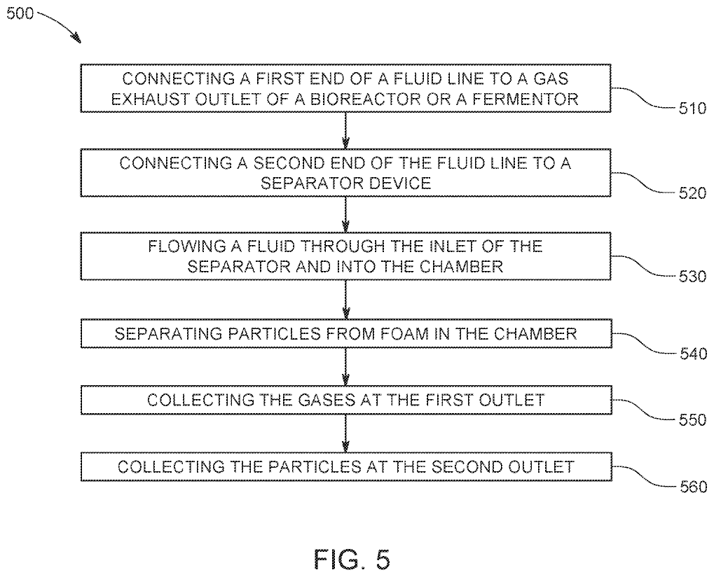

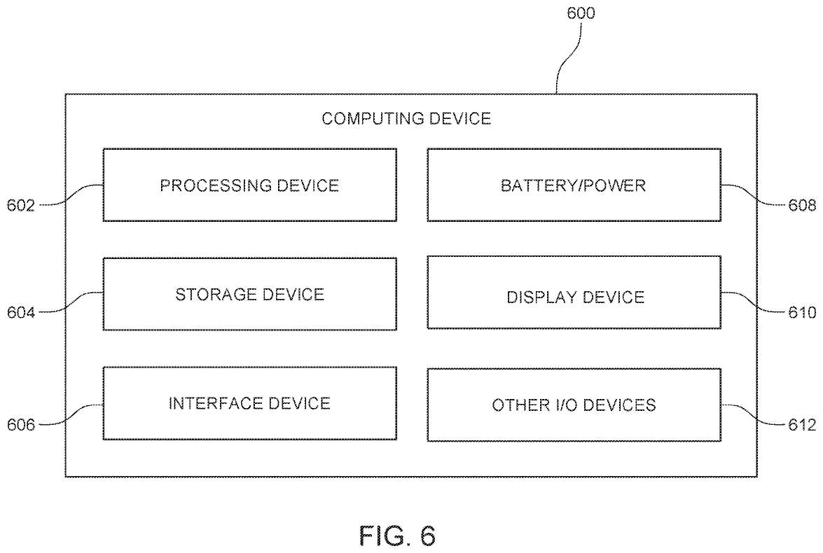

FIG. 5 is a flow diagram of method 500 for operating separator assembly 250, in accordance with the embodiments described above. Aspects of the example separator assemblies 150, 250, and 350, which are depicted in FIGS. 1A, 1B, 2, and 3A-C, can be utilized in the method steps described below. Example method 500 herein described may not recite the complete process or all steps of the method. Also, all steps need not necessarily be performed, and in some cases, the steps can be performed simultaneously, the steps can be performed in a different order than the order shown, additional steps can be included at each step, or original steps can be replaced with alternate steps.

At step 510, a first end 140A of a fluid line 140 is connected to a gas exhaust outlet 138B of a bioreactor or a fermentor 120. Fluid line 140 is configured to receive and allow flow of multiphase fluid 105 generated by bioreactor 120 during the cultivation of cells. Multiphase process fluid 105 from bioreactors mainly comprises unreacted gaseous matter, particulate matter, moisture/liquids, and foam. Step 510 can further include monitoring of initiation of fluid flow out of exhaust outlet 138B of bioreactor 120, by system controller 180, to control the operation of valves and pumps on fluid line 140 to push the multiphase fluid 105 to separator assembly 250 as described below.

At step 520, a second end 140B of fluid line 140 is connected to a separator device 250. Separator device 250 includes a separator body 255 having a chamber 259 with a central axis C therein. An inlet 248 is connected to separator body 255 and is in fluidic communication with first fluid line 140, and inlet 248 is configured to allow multiphase process fluid 105, to flow into chamber 259 tangentially at speeds of at least 500 Lpm. Further, first and second outlets 252, 254 connected to separator body 255, are in fluidic communication with chamber 259.

At step 530, multiphase process fluid 105 is flowed through inlet 248 of separator 250 and into chamber 259. Receiving fluid flow at appropriate speeds (at least 500 Lpm) into chamber 259 in a tangential direction triggers the formation of a vortex motion 215 of the multiphase process fluid 105. In other words, multiphase process fluid 105 moves around in chamber 259 in a helical or spiral pattern around central axis C to generate a cyclonic effect.

At step 540, the light phase is separated from the heavy phase in chamber 259. As a result of vortex motion 215 of the fluid flow inside chamber 259, foam is broken down to release particulate matter, and light particles are separated from heavy particles. Additionally, step 540 can include accumulating gases free of particulate matter proximate to central axis C. Likewise, step 540 can also include, propelling the particulate matter away from central axis C towards the inner wall of frustoconical lower portion 257.

At step 550, exhaust gases free of particulate matter or including only light particles are collected at first outlet 252. For example, exhaust gases from first outlet 252 are more than 90% free from particulate matter and moisture. When passed through exhaust unit 156, these exhaust gases are passed out freely without clogging the filters, thus increasing the life of exhaust filters.

At step 560, particle matter is collected at second outlet 254. Particle matter can include particulate matter categorized based on particle sized beyond a threshold size and are collected at second outlet 254. Step 560 can include flowing the particle matter down fluid line 162 into holding tank 160. Further step 560 can include pushing the particle matter back into container 122 by fluid line 165 through inlet port 138D if recycling is required. Optionally step 560 can include collecting particle matter at waste receiver 164 by waste line 163 for disposal if recycling is not required.

Referring back to FIGS. 1A, and 1B, and as discussed previously, automated bioprocessing system 100 includes controlling one or more operations of separator assembly 150 to purify multiphase process fluid 105 from bioreactor 120. In example embodiments, during cultivation of cells in bioprocess fluid 126 in container 122, sensor 128A can provide data for temperature characteristics of bioprocess fluid 126 with time to sensor interface module 188, which can be saved as data set S1 in memory 184 of system controller 180. Similarly, sensor 128B can provide data for pH variation for bioprocess fluid 126 with time to sensor interface module 188, which can be saved as data set S2 in memory 184 of system controller 180. Similarly, sensor 128C can provide data for foam buildup above bioprocess fluid 126 with time to sensor interface module 188, which can be saved as data set S3 in memory 184 of system controller 180. System controller 180 can be configured to automatically process data sets S1, S2, and S3 through processor 182, and to prepare instructions for the operation of equipment (valves 144 and pumps 146). For example, if foam build-up in container 122 exceeds a threshold value, system controller 180 can give instructions to open release valve 144A and initiate operation of pump 146A so that multiphase process fluid 105 from container 122 is vented out through gas outlet 138B and through fluid line 140 to inlet 148 of separator assembly 150 at appropriate speeds. Alternatively, if foam build-up in container 122 exceeds a threshold value, system controller 180 can give instructions to antifoam dispenser 137 to dispense antifoam into chamber 122 or control sparging of gas through spargers 130A, 130B. To further optimize the operation of valves and pumps, system controller can retrieve data from sensor 128D disposed on fluid line 140. Sensor 128D provides a more current and accurate flow status of multiphase process fluid 105 in fluid line 140.

System controller 180 is further configured to control pump 146A so that the multiphase fluids 105 are flowed into separator assembly 150 at appropriate speeds to enhance separation characteristics in separator assembly 150. Upon sensing of a fluid flow through fluid lines 158 by sensors 128H, system controller 180 registers a ‘separation process complete’ status for the particle separation stage and can give instructions (i) to open release valve 144B and initiate operation of pump-2 146B so that the light phase, including exhaust gases vented out through first outlet 152 of separator assembly 150, are further vented out through exhaust unit 156; and (ii) to open release valve 144C so that the heavy phase, including liquid and/or particulate matter collected at second outlet 154 of separator assembly 150, is collected in holding tank 160 through fluid line 162.

Sensors 128D, 128E, 128F coupled to holding tank 160 can provide feedback to system controller 180 with respect to contents received in holding tank 160. In case of failed operation at separator assembly 150, the contents of holding tank 160 may include excessive amounts of valuable product or biocomponents. System controller 180 can access foam layer measurements provided by sensor 128D to determine if foam-out has occurred in holding tank 160. System controller 180 can access cell density measurements provided by sensor 128E to determine if the cell density of particle matter in holding tank 160 is beyond a threshold value. Further system controller 180 can access Raman or Mass spectroscopic measurements provided by sensor 128F to identify the composition of contents of holding tank 160. Based on feedback from sensors 128D-F, if it is determined that holding tank includes valuable product or biocomponents, system controller 180 can control operation of valve 144D and pump-3 146C to pump back the contents of holding tank 160 back to chamber 122 by fluid line 165. Likewise, based feedback from sensors 128D-F, if it is determined that holding tank does not include valuable biocomponents, system controller 180 can control operation of valve 144E to allow flow of content of holding tank 160 into waste receiver 164 by fluid line 163.

Foam sensor 128I coupled to separator assembly 150 provides for measuring foam height, foam volume, and foam concentration in separator assembly 150 during the separation process. System controller 180 can access the above foam parameters in separator assembly 150 to control foam generation in bioreactor 120 by activating anti-foam dispenser 137. Alternatively, system controller 180 can control the speed of impeller 132 or the operation of valve 144A, pump-1 146A to regulate the entry of multiphase fluid 105 into separator assembly 150.

Further, system controller 180 can monitor particle count data generated by particle counter 170 on fluid line 168 to analyze the quality of purification, and quantity of gases exhausted by exhaust unit 156, as described in detailed experimental results below.

A user operating the user workstation 190 through a graphical user interface 192 is equipped to control operations of equipment of separator system 150 through components of system controller 180. Users can also gather data sets related to separator system 150 to prepare reports and data useful for scaleup operations.

In an example, a 30 L Single Use Fermentor (SUF) was used for running a control experiment, including E. Coli fermentation. The below table shows the results of particle count measured based on particle sizes in the exhaust stream with and without using separator assembly 250 described above. Column A lists particle count measured based on particle size, when separator assembly 250 was not used. Column B lists particle count measured based on particle size, when separator assembly 250 including a radius R1 (radius of upper portion 253 of separator assembly 250) of two inches was used. Column C lists the efficiency of particle separation based on particle size. The results below show that usage of separator assembly 250 reduced particle count in the exhaust stream by 92% with 100% separation for particle sizes ranging between 5.0 to 10.0 uM Δ (N)/s.

| | Column A | Column B | Column C |
|---|---|---|---|
| Test # | No cyclone | 2-inch cyclone | Efficiency % |
| Test Medium | E. Coli | E. Coli | |
| Vessel | 30 L SUF | 30 L SUF | |
| Test Medium Temp (°C) | 37 | 37 | |
| Flow rate 1 (lpm) | 100 | 100 | |
| Avg. 0.5 uM Δ (N)/s | 53,900.95 | 10,113.59 | 81 |
| Avg. 1.0 uM Δ (N)/s | 51,251.53 | 2,715.21 | 95 |
| Avg. 2.0 uM Δ (N)/s | 47,787.97 | 472.58 | 99 |
| Avg. 5.0 uM Δ (N)/s | 12,714.54 | 0.02 | 100 |
| Avg. 10.0 uM Δ (N)/s | 175.65 | 0.01 | 100 |
| Avg. 25.0 uM Δ (N)/s | 0.01 | 0.01 | 0 |
| Total | 165,830.65 | 13,301.42 | 92 |

FIG. 6 depicts a block diagram of an example computing device 600 that can perform some or all operations of an automated bioprocessing system, including a separator system, user computing device(s), processing unit(s) and/or controller(s) in accordance with the example embodiments. The example automated bioprocessing system, including a separator system, and system controller, including controllers, modules, libraries, and data repositories, disclosed herein can include or be implemented by one or more computing devices. In some embodiments, the example user computing device or workstation 190, and system controller 180 include a single computing device 600 or multiple computing devices 600. Further, as discussed below, a computing device 600 (or multiple computing devices 600) that implements the example automated bioprocessing system, including a separator system, modules, data repositories, and libraries, can be part of one or more separator assemblies 150, user or client computing devices 190 with user interfaces 192, processors 182 and controllers 180, a user's local computing device, a service provider's local computing device, or a remote computing device. Client computing devices or user workstations 190, processing units 182, and controllers 180, can also be contained in a unitary computing system or server with a user interface or distributed over servers and systems.

The computing device 600 of FIG. 6 is illustrated as having a number of components, but any one or more of these components may be omitted or duplicated, as suitable for the application and setting. In some embodiments, some or all of the components included in the computing device 800 can be attached to one or more motherboards and enclosed in a housing (e.g., including plastic, metal, and/or other materials). In some embodiments, some of these components may be fabricated onto a single system-on-a-chip (SoC) (e.g., an SoC may include one or more processing devices 602 and one or more storage devices 604). Additionally, in various embodiments, the computing device 600 may not include one or more of the components illustrated in FIG. 6, but may include interface circuitry (not shown) for coupling to one or more components using any suitable interface (e.g., a Universal Serial Bus (USB) interface, a High-Definition Multimedia Interface (HDMI) interface, a Controller Area Network (CAN) interface, a Serial Peripheral Interface (SPI) interface, an Ethernet interface, a wireless interface, or any other appropriate interface). For example, the computing device 600 may not include a display device 610, but may include display device interface circuitry (e.g., a connector and driver circuitry) to which a display device 610 may be coupled.

The computing device 600 can include a processing medium or device 602 (e.g., one or more processing devices). As used herein, the term “processing device” refer to any device or portion of a device that processes electronic data from registers and/or memory to transform that electronic data into other electronic data that may be stored in registers and/or memory. The processing device 602 can include one or more digital signal processors (DSPs), application-specific integrated circuits (ASICs), central processing units (CPUs), graphics processing units (GPUs), crypto processors (specialized processors that execute cryptographic algorithms within hardware), server processors, or any other suitable processing devices.

The computing device 600 can also include a storage device 604 (e.g., one or more storage devices). The storage device 604 can include one or more memory devices such as random-access memory (RAM) (e.g., static RAM (SRAM) devices, magnetic RAM (MRAM) devices, dynamic RAM (DRAM) devices, resistive RAM (RRAM) devices, or conductive-bridging RAM (CBRAM) devices), hard drive-based memory devices, solid-state memory devices, networked drives, cloud drives, or any combination of memory devices. In some embodiments, the storage device 604 can include memory that shares a die with a processing device 602. In such an embodiment, the memory can be used as cache memory and can include embedded dynamic random-access memory (eDRAM) or spin transfer torque magnetic random-access memory (STT-MRAM), for example. In some embodiments, the storage device 604 can include non-transitory computer-readable media having instructions thereon that, when executed by one or more processing devices (e.g., the processing device 602), cause the computing device 600 to perform any appropriate ones of or portions of the methods and operations disclosed herein.

The computing device 600 can include an interface device 606 (e.g., one or more interface devices 606). The interface device 606 can include one or more communication chips, connectors, and/or other hardware and software to govern communications between the computing device 600 and other computing devices. For example, the interface device 606 can include circuitry for managing wireless communications for the transfer of data to and from the computing device 600. The term “wireless” and its derivatives are used to describe circuits, devices, systems, methods, techniques, communications channels, etc., that can communicate data through the use of modulated electromagnetic radiation through a nonsolid medium. The term does not imply that the associated devices do not contain any wires, although in some embodiments they might not. Circuitry included in the interface device 606 for managing wireless communications can implement any of a number of wireless standards or protocols, including but not limited to Institute for Electrical and Electronic Engineers (IEEE) standards including Wi-Fi (IEEE 802.11 family), IEEE 802.16 standards (e.g., IEEE 802.16-2005 Amendment), Long-Term Evolution (LTE) project along with any amendments, updates, and/or revisions (e.g., advanced LTE project, ultra-mobile broadband (UMB) project (also referred to as “3GPP2”), etc.). In some embodiments, circuitry included in the interface device 606 for managing wireless communications may operate in accordance with a Global System for Mobile Communication (GSM), General Packet Radio Service (GPRS), Universal Mobile Telecommunications System (UMTS), High Speed Packet Access (HSPA), Evolved HSPA (E-HSPA), or LTE network. In some embodiments, circuitry included in the interface device 806 for managing wireless communications can operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). In some embodiments, circuitry included in the interface device 606 for managing wireless communications can operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. In some embodiments, the interface device 606 can include one or more antennas (e.g., one or more antenna arrays) to receipt and/or transmission of wireless communications.

In some embodiments, the interface device 606 can include circuitry for managing wired communications, such as electrical, optical, or any other suitable communication protocols. For example, the interface device 606 can include circuitry to support communications in accordance with Ethernet technologies. In some embodiments, the interface device 606 can support both wireless and wired communication, and/or may support multiple wired communication protocols and/or multiple wireless communication protocols. For example, a first set of circuitries of the interface device 606 can be dedicated to shorter-range wireless communications such as Wi-Fi or Bluetooth, and a second set of circuitries of the interface device 606 can be dedicated to longer-range wireless communications such as global positioning system (GPS), EDGE, GPRS, CDMA, WiMAX, LTE, EV-DO, or others. In some embodiments, a first set of circuitries of the interface device 606 can be dedicated to wireless communications, and a second set of circuitries of the interface device 606 can be dedicated to wired communications.

The computing device 600 can include battery/power circuitry 608. The battery/power circuitry 608 can include one or more energy storage devices (e.g., batteries or capacitors) and/or circuitry for coupling components of the computing device 600 to an energy source separate from the computing device 600 (e.g., AC line power).

The computing device 600 can include a display device 610 (e.g., multiple display devices). The display device 610 can include any visual indicators, such as a heads-up display, a computer monitor, a projector, a touchscreen display, a liquid crystal display (LCD), a light-emitting diode display, or a flat panel display.

The computing device 600 can include other input/output (I/O) devices 612. The other I/O devices 612 can include one or more audio output devices (e.g., speakers, headsets, earbuds, alarms, etc.), one or more audio input devices (e.g., microphones or microphone arrays), location devices (e.g., GPS devices in communication with a satellite-based system to receive a location of the computing device 600, as known in the art), audio codecs, video codecs, printers, sensors (e.g., thermocouples or other temperature sensors, humidity sensors, pressure sensors, vibration sensors, accelerometers, gyroscopes, etc.), image capture devices such as cameras, keyboards, cursor control devices such as a mouse, a stylus, a trackball, or a touchpad, bar code readers, Quick Response (QR) code readers, or radio frequency identification (RFID) readers, for example.

The computing device 600 can have any suitable form factor for its application and setting, such as a handheld or mobile computing device (e.g., a cell phone, a smartphone, a mobile internet device, a tablet computer, a laptop computer, a netbook computer, an Ultrabook computer, a personal digital assistant (PDA), an ultra-mobile personal computer, etc.), a desktop computing device, or a server computing device or other networked computing components.

The different embodiments and examples of the separator systems and methods described herein provide several advantages over known solutions for purifying exhaust gases from bioreactors. For example, illustrative embodiments and examples described herein allow for the smooth operation of multiphase fluid purification by automation, especially in cases where multiphase fluid include foam and particulate matter of varying sizes.

Additionally, and among other benefits, illustrative embodiments and examples described herein allow for reducing cost and time for process optimization of purification of exhaust gases.

Additionally, and among other benefits, illustrative embodiments and examples described herein prevent the clogging of exhaust filters, thereby increasing the lifetime of filters.

Additionally, and among other benefits, illustrative embodiments and examples described herein are configured to provide a complete, single-use solution for multiphase fluid purification in downstream bioprocessing of recombinant proteins such as monoclonal antibodies and viral vector production.

Additionally, and among other benefits, illustrative embodiments and examples described herein allow for using separators connected in parallel or series to enhance the purification of multiphase fluid.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, processes, and/or products according to certain embodiments of the present disclosure may include, incorporate, or otherwise comprise properties features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting the application or inclusion of said features to the specific embodiment. Rather, it will be appreciated that other embodiments can also include said features without necessarily departing from the scope of the present disclosure.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

1. A multiphase fluid separator, comprising:
a separator body having an upper portion and a lower portion, a chamber therein configured to break a foam and release a solid or a gas entrapped in the foam, and a central axis extending from the upper portion to the lower portion and through the chamber;
an inlet connected to the separator body and in fluidic communication with a fluid line connected to a bioreactor or a fermenter, the inlet configured to direct a multiphase fluid comprising the foam through the fluid line and tangentially into the chamber;
a first outlet and a second outlet in fluidic communication with the chamber; and
an exhaust line having a first end and an opposing second end, the first end of the exhaust line in fluidic communication with the first outlet.

2. The multiphase fluid separator as recited in claim 1, further comprising an exhaust filter unit connected to the second end of the exhaust line, the exhaust filter unit configured to filter a microbe from the gas.

3. The multiphase fluid separator as recited in claim 1, wherein the upper portion of the separator body has a cylindrical wall structure, and the lower portion has a frustoconical wall structure.

4. The multiphase fluid separator as recited in claim 3, wherein a diameter of the frustoconical wall structure of the lower portion tapers in a direction away from the upper portion.

5. The multiphase fluid separator as recited in claim 3, wherein the inlet is aligned tangential to the cylindrical wall structure of the upper portion, and the first and second outlets are aligned along the central axis.

6. The multiphase fluid separator as recited in claim 1, wherein the first outlet includes a tubular extension extending into the upper portion of the separator body and inside the chamber.

7. The multiphase fluid separator as recited in claim 1, wherein a spatial alignment of the inlet, first and second outlets, with the upper and lower portions forces the multiphase fluid into a vortex motion through the chamber and around the central axis.

8. The multiphase fluid separator as recited in claim 7, wherein the vortex motion of the multiphase fluid breaks the foam and releases the solid or the gas in the foam.

9. The multiphase fluid separator as recited in claim 1, wherein the chamber comprises a spiral pathway for forcing the multiphase fluid into a vortex motion through the chamber.

10. The multiphase fluid separator as recited in claim 8, wherein the solid comprises cell debris, surfactants, proteinaceous materials, poloxamer materials, polyethylene glycol or algae.

11. The multiphase fluid separator as recited in claim 1, further comprising a holding tank fluidically connected to the second outlet, the holding tank configured to collect the solid.

12. The multiphase fluid separator as recited in claim 11, further comprising a foam sensor, level sensor, Raman sensor or cell density sensor coupled to the holding tank.

13. A method for separating a multiphase fluid, comprising:
flowing the multiphase fluid comprising foam with entrapped solid and gas through a first separator assembly comprising:
a separator body having a chamber with a central axis, an inlet, a first outlet, a second outlet and a fluid pathway through the chamber configured to break the foam and route the gas through the first outlet and solid through the second outlet.

14. The method as recited in claim 13, wherein the separator body comprises an upper portion and a lower portion, the inlet and the first outlet are connected to the upper portion, and the second outlet is connected to the lower portion.

15. The method as recited in claim 13, wherein flowing the multiphase fluid through the first separator assembly causes the multiphase fluid to move in a vortex motion in the chamber.

16. The method as recited in claim 13, wherein the fluid pathway is in a spiral shape and causes the gas to accumulate around the central axis.

17. The method as recited in claim 16, wherein the spiral shaped fluid pathway causes the solid to propel away from the central axis.

18. The method as recited in claim 17, further comprising flowing the gas to an exhaust filter through an exhaust line connected to the second outlet.

19. The method as recited in claim 13, wherein the solid comprises particles of different sizes.

20. The method as recited in claim 13, further comprising replacing the first separator assembly with a second separator assembly for a subsequent batch of separating the multiphase fluid.

21.-33. (canceled)
